(19) United States
(12) Patent Application Publication
(10) Pub. No.: US 2024/0417792 A1
(43) Pub. Date: Dec. 19, 2024

(54) CAPTURING AND AMPLIFYING POLYNUCLEOTIDES, AND REUSING FLOWCELLS, USING SILICA PARTICLES

(71) Applicant: Illumina, Inc., San Diego, CA (US)

(72) Inventors: Angelo La Rosa, Biggleswade (GB); Xavier von Hatten, Cambridge (GB)

(73) Assignee: Illumina, Inc., San Diego, CA (US)

(21) Appl. No.: 18/736,835

(22) Filed: Jun. 7, 2024

Related U.S. Application Data
(60) Provisional application No. 63/507,029, filed on Jun. 8, 2023.

(51) Int. Cl. C12Q 1/6874; C12Q 1/6806; C12Q 1/6834; C12Q 1/6844

(52) U.S. Cl. CPC C12Q 1/6874; C12Q 1/6806; C12Q 1/6834; C12Q 1/6844

(57) ABSTRACT

In some examples, a method of sequencing a polynucleotide includes hybridizing the polynucleotide to a first oligonucleotide coupled to a particle. The hybridized polynucleotide may be amplified using additional oligonucleotides coupled to the particle, to generate amplicons coupled to the particle. After the amplifying, the particle, having the amplicons coupled thereto, may be disposed within a flowcell. After disposing the particle within the flowcell, the particle may be dissolved, leaving the amplicons within the flowcell. The flowcell may be used to sequence the amplicons.

### CROSS-REFERENCE TO RELATED APPLICATIONS

This application claims the benefit of U.S. Provisional Patent Application No. 63/507,029, filed Jun. 8, 2023 and entitled “Capturing and Amplifying Polynucleotides, and Reusing Flowcells, Using Silica Particles,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2437-US.xml”, was created on May 15, 2024 and is 12 kB in size.

### FIELD

This application generally relates to capturing and amplifying polynucleotides.

### BACKGROUND

Cluster amplification is an approach to amplifying polynucleotides, for example for use in genetic sequencing. Target polynucleotides are captured by primers (e.g., P5 and P7 primers) coupled to a substrate surface in a flowcell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to capturing and amplifying polynucleotides, and reusing flowcells, using silica particles. Particles for performing such capture, amplification, and flowcell reuse, and methods of making such particles, also are disclosed.

Some examples herein provide a method of sequencing a polynucleotide. The method may include hybridizing the polynucleotide to a first oligonucleotide coupled to a particle. The method may include amplifying the hybridized polynucleotide using additional oligonucleotides coupled to the particle, to generate amplicons coupled to the particle. The method may include, after the amplifying, disposing the particle, having the amplicons coupled thereto, within a flowcell. The method may include, after disposing the particle within the flowcell, dissolving the particle, leaving the amplicons within the flowcell. The method may include using the flowcell to sequence the amplicons.

In some examples, disposing the particle, having the amplicons coupled thereto, within the flowcell includes electrostatically attracting the particle or the amplicons to a region of the flowcell.

In some examples, the region of the flowcell includes a recess. In some examples, the recess includes a hydrogel. In some examples, the recess includes a plurality of positively charged moieties.

In some examples, the particle is dissolved while using the flowcell to sequence the amplicons. In some examples, the particle is dissolved before using the flowcell to sequence the amplicons.

In some examples, the oligonucleotides are coupled to a polymer that is coupled to the particle. In some examples, dissolving the particle further leaves the polymer within the flowcell, the amplicons being coupled to the polymer left within the flowcell. In some examples, the method further includes, after sequencing the amplicons, removing the polymer and the amplicons. In some examples, the polymer and the amplicons are removed using a basic solution. In some examples, the polymer is coupled to the particle via a siloxane (—Si—O—Si—) bond. In some examples, the polymer includes polyacrylamide. In some examples, before the particle is dissolved, the polymer is covalently coupled to the particle. In some examples, both before and after the particle is dissolved, the polymer is covalently coupled to the amplicons. In some examples, both before and after the particle is dissolved, the polymer is non-covalently coupled to the flowcell.

In some examples, the particle includes silica. In some examples, the particle is dissolved using a basic solution. In some examples, the particle consists essentially of silica.

Some examples herein provide a method of sequencing a polynucleotide using a particle having a plurality of oligonucleotides coupled thereto. The method may include using the plurality of oligonucleotides to generate amplicons of the polynucleotide that are coupled to the particle. The method may include disposing the particle, having the amplicons coupled thereto, within a flowcell. The method may include, after disposing the silica particle within the flowcell, dissolving the particle. The method may include using the flowcell to sequence the amplicons.

Some examples herein provide a method of using a flowcell. The method may include disposing a first particle, having a first set of polynucleotide amplicons coupled thereto, within the flowcell. The method may include dissolving the first particle, leaving the first set of polynucleotide amplicons within the flowcell. The method may include using the flowcell to sequence the first set of polynucleotide amplicons. The method may include, after sequencing the first set of polynucleotide amplicons, removing the first set of polynucleotide amplicons from the flowcell. The method may include, after removing the first set of polynucleotide amplicons from the flowcell, disposing a second particle, having a second set of polynucleotide amplicons coupled thereto, within the flowcell.

In some examples, the method further includes dissolving the second particle, leaving the second set of polynucleotide amplicons within the flowcell. The method further may include using the flowcell to sequence the second set of polynucleotide amplicons. The method further may include, after sequencing the second set of polynucleotide amplicons, removing the second set of polynucleotide amplicons from the flowcell. The method further may include, after removing the second set of polynucleotide amplicons from the flowcell, disposing a third particle, having a third set of polynucleotide amplicons coupled thereto, within the flowcell.

Some examples herein provide a composition that includes a particle coupled to a plurality of polynucleotide amplicons; and a fluid in contact with the particle coupled to the plurality of polynucleotide amplicons, the fluid including a reagent in a concentration suitable to substantially dissolve the particle, substantially without damaging the polynucleotide amplicons.

Some examples herein provide a device that includes a flowcell, and a particle disposed within the flowcell. The particle may be coupled to a plurality of polynucleotide amplicons. The device further may include a reservoir storing a fluid including a reagent in a concentration suitable to substantially dissolve the particle, substantially without damaging the polynucleotide amplicons. The device further may include a fluidic pathway coupling the reservoir to the flowcell. The device further may include circuitry configured to cause the fluid to flow from the reservoir to the flowcell so as to contact, and substantially dissolve, the particle, leaving the polynucleotide amplicons within the flowcell.

Some examples herein provide a method of disposing a polymer in a flowcell. The method may include disposing a particle, coupled to the polymer, within the flowcell; and dissolving the particle, leaving the polymer within the flowcell.

Some examples herein provide a composition that includes a particle coupled to a polymer; and a fluid in contact with the particle coupled to the polymer. The fluid may include a reagent in a concentration suitable to substantially dissolve the particle, substantially without damaging the polymer.

Some examples herein provide a device that includes a flowcell; and a particle disposed within the flowcell. The particle may be coupled to a polymer. The device further may include a reservoir storing a fluid including a reagent in a concentration suitable to substantially dissolve the particle, substantially without damaging the polymer. The device further may include a fluidic pathway coupling the reservoir to the flowcell. The device further may include circuitry configured to cause the fluid to flow from the reservoir to the flowcell so as to contact, and substantially dissolve, the particle, leaving the polymer within the flowcell.

Some examples herein provide a method of modifying a silica particle. The method may include contacting the silica particle with a first molecule. The first molecule may include a silane coupled to a first functional group. The silane reacts with a silanol (—Si—OH) group of the silica particle to form a siloxane (—Si—O—Si—) bridge via which the first functional group is coupled to the silica particle. The method may include contacting the silica particle with a second molecule. The second molecule may include a polymer coupled to second and third functional groups. The first functional group reacts with the second functional group to form a bond via which the polymer is coupled to the silica particle. The method may include contacting the silica particle with a third molecule. The third molecule may include an oligonucleotide coupled to a fourth functional group. The third functional group reacts with the fourth functional group to form a bond via which the oligonucleotide is coupled to the silica particle.

In some examples, the silane includes a trialkoxy silane.

In some examples, the second and third functional groups are of the same type as one another. In some examples, the second and third functional groups are of different types than one another.

In some examples, the polymer includes polyacrylamide.

In some examples, the silica particle has a diameter of about 10 nm to 1 μm.

In some examples, the oligonucleotide includes an amplification primer.

Some examples herein provide a method of modifying a silica particle coupled to a first functional group via a siloxane (—Si—O—Si—) bridge. The method may include contacting the silica particle with a first molecule. The first molecule may include a polymer coupled to second and third functional groups. The first functional group reacts with the second functional group to form a bond via which the polymer is coupled to the silica particle. The method may include contacting the silica particle with a second molecule, the second molecule including an oligonucleotide coupled to a fourth functional group. The third functional group reacts with the fourth functional group to form a bond via which the oligonucleotide is coupled to the silica particle.

Some examples herein provide a method of modifying a silica particle coupled to a first functional group via a siloxane (—Si—O—Si—) bridge and a polymer. The method may include contacting the silica particle with a molecule. The molecule may include an oligonucleotide coupled to a second functional group. The third functional group reacts with the fourth functional group to form a bond via which the oligonucleotide is coupled to the silica particle.

Some examples herein provide a composition made using any one of the foregoing methods.

Some examples herein provide a composition that includes a silica particle; and a molecule coupled to the silica particle via a siloxane (—Si—O—Si—) bridge. The molecule may include a polymer coupled to the siloxane via a reaction product of first and second functional groups. The composition may include a plurality of oligonucleotides coupled to the polymer via reaction products of third and fourth functional groups.

Some examples herein provide a method of using such a composition. The method may include hybridizing a template polynucleotide to a first oligonucleotide of the plurality. The method may include amplifying the hybridized template polynucleotide using additional oligonucleotides of the plurality to generate a plurality of amplicons coupled to the polymer via reaction products of third and fourth functional groups.

In some examples, the method further includes, after the amplifying, disposing the silica particle within a flowcell. In some examples, disposing the silica particle within the flowcell includes electrostatically attracting the silica particle to a region of the flowcell. In some examples, the method further includes, after disposing the silica particle within the flowcell, dissolving the silica particle.

Some examples herein provide a device including a flowcell; and any one of the foregoing compositions located within the flowcell.

In some examples, the flowcell includes a positively charged surface to which the composition is noncovalently bound through an electrostatic force.

Some examples herein provide a composition that includes a silica particle; and a molecule coupled to the silica particle via a siloxane (—Si—O—Si—) bridge. The molecule may include a polymer coupled to the siloxane via a reaction product of first and second functional groups. The particle may include a plurality of polynucleotide amplicons coupled to the polymer via reaction products of third and fourth functional groups.

Some examples herein provide a device including: a flowcell; and any one of the foregoing compositions located within the flowcell.

In some examples, the flowcell includes a positively charged surface to which the composition is noncovalently bound through an electrostatic force.

Some examples herein provide a method of using any one of the foregoing devices. The method may include dissolving the silica particle within the flowcell.

Some examples herein provide a method of using any one of the foregoing devices. The method may include sequencing the plurality of polynucleotide amplicons within the flowcell.

Some examples herein provide a method of making a silica particle. The method may include reacting a silicon precursor with ammonium hydroxide to form a silica particle including a plurality of silanol (—Si—OH) groups. The method may include, without washing the silica particle after forming it, contacting the silica particle with a molecule including a silane coupled to a functional group, wherein the silane reacts with the silanol group to form a siloxane (—Si—O—Si—) bridge via which the functional group is coupled to the silica particle.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-IF schematically illustrate example structures and operations for preparing a silica particle coupled to a polymer, and optionally also to oligonucleotides.

FIGS. 2A-2N schematically illustrate example structures and operations for capturing and amplifying a polynucleotide, and optionally also reusing a flowcell, using a silica particle.

FIG. 3 illustrates a plot of the sizes, measured using dynamic light scattering (DLS), of example silica particles prepared according to certain examples.

FIGS. 4A-4B illustrate plots of the measured size distributions of example silica particles prepared according to certain examples.

FIGS. 5A-5B illustrate fluorescence signals obtained from a flowcell during sequencing operations performed using example silica particles.

FIGS. 6A-6B respectively illustrate scanning electron microscopy (SEM) images of the top and bottom of a flowcell after sequencing operations performed using example silica particles.

FIG. 7A illustrates a plot of the size distributions, measured using DLS, of example silica particles before and after exposure to a dissolving reagent.

FIG. 7B illustrates a correlogram of example silica particles before and after exposure to a dissolving reagent.

### DETAILED DESCRIPTION

Examples provided herein are related to capturing and amplifying polynucleotides, and reusing flowcells, using silica particles. Silica particles for performing such capture, amplification, and flowcell reuse, and methods of making such particles, also are disclosed.

Some examples herein relate to silica particles and their methods of preparation and use. Such particles optionally may be used to capture and amplify a polynucleotide in solution, and the particles (with amplicons of the polynucleotide attached) subsequently may be disposed on a flowcell for use in sequencing the amplicons, e.g., using sequencing-by-synthesis. Because the particles capture selectively within the nanowells, the construction of the flowcell may be significantly simplified relative to that of a flowcell that is coated and subsequently polished to remove the material sticking at the interstitials between the nanowells; for example, a flowcell for use with the present particles need not necessarily include depression patterned regions of capture primers, as may be the case for flowcells fabricated with previously known technology. After the particle captures the polynucleotide, amplification is performed at the surface of the particle where all the strands of a cluster are confined, and the particle may be disposed at any suitable location on the flowcell. Additionally, monoclonality of the clusters may be improved by performing seeding and clustering using particles in solution, for example because the particles may be expected to be sufficiently spaced apart from one another in the solution that a cluster of amplicons coupled to a given particle may not be expected to be able to be contaminated by amplicons coupled to another such particle; in comparison, performing seeding and clustering on a flowcell surface runs the risk that an amplicon from one cluster may contaminate another cluster, resulting in generation of a polyclonal cluster which may not be usable for sequencing. Additionally, the present particles may increase the speed with which sequencing may be performed, for example because the capture and amplification operations may be performed off-instrument without the need to utilize time on the instrument to perform such operations; in comparison, performing capture and amplification on the flowcell can utilize significant instrument time that otherwise could have been used for sequencing.

First, some terms used herein will be briefly explained. Then, some example particles and example methods for capturing and amplifying a polynucleotide, for reusing a flowcell using silica particles, and methods of making silica particles, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to +10%, such as less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another. Polynucleotides that are “partially” hybridized to one another means that they have sequences that are complementary to one another, but such sequences are hybridized with one another along only a portion of their lengths to form a partial duplex. Polynucleotides with an “inability” to hybridize include those which are physically separated from one another such that an insufficient number of their bases may contact one another in a manner so as to hybridize with one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosinc diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to a substrate and may hybridize to an adapter of the target polynucleotide. In some cases, a capture primer that is coupled to the substrate and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the substrate, but that may not be used to grow a complementary strand during an amplification process, may in some cases be referred to as a “seeding primer.” A capture primer that may be used to grow a complementary strand during an amplification process may in some cases be referred to as an “amplification primer.”

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kchagias et al., Microelectronic Engineering 86 (2009), pp. 776-778, which is incorporated by reference in its entirety. In some examples, substrates used in the present application include silica-based substrates, such as glass, fused silica, or other silica-containing material. As used herein, by “silica” it is meant silicon dioxide with chemical composition of SiOₓ, where x is approximately equal to 2. Silica may be porous. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible.

In some examples, a substrate is or includes a particle. As used herein, a “particle” is a small, localized object which exists as a discrete unit in a given medium. In detail, the term refers to microscopic particles with sizes ranging from atoms to molecules, such as nanoparticles or colloidal particles. A particle may refer to a substrate that is sufficiently small that it may be located within a fluid (such as a liquid), that is, substantially surrounded on all sides by the fluid. As such, a particle may be carried by (moved by) the fluid in which it is located, for example by flowing the fluid from one location to another location. As explained elsewhere herein, particles can have a variety of shapes. Optionally, particles may be porous. When a particle is at least partially spherical, it may be referred to as a “bead.” In some examples, a first substrate (such as a flat or patterned surface within a flowcell) may be used to support a second substrate (such as a particle).

In some examples, a substrate includes a patterned surface. A “patterned surface” refers to an arrangement of different regions in or on an exposed layer of a substrate. For example, one or more of the regions may include feature(s) that can be used to attract and/or be coupled to a particle. The feature(s) can be separated by interstitial regions where such features are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface, into which particles respectively may be disposed. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM, which is a type of polyacrylamide). The process creates hydrogel regions used to attract particles for sequencing that may be stable over sequencing runs with a large number of cycles. In some examples, covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA, which is another type of polyacrylamide) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells. The azido groups of the hydrogel material may be converted to, or coupled to, groups with a positive charge. A solution including the present particles may then be contacted with the polished substrate such that individual target particles may become attracted to, and disposed within, individual wells via interactions with the positively charged groups attached to the hydrogel material; however, the particles substantially will not occupy the interstitial regions due to absence or inactivity of the hydrogel material. Before or after contacting the particles with the substrate, the particles may be used to capture and amplify a plurality of target polynucleotides (e.g., a fragmented human genome or portion thereof). Amplicons of the target polynucleotides may be confined to the wells because the particles are confined to the wells. The amplicons then may be sequenced. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flowcell or is located in or coupled to a flowcell. Flowcells may include a flow chamber that is divided into a plurality of lanes or a plurality of sectors. Example flowcells and substrates for manufacture of flowcells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate. A hydrogel may be located on a substrate, such as on a particle or on a region of a flowcell.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate is intended to mean that the layer covers the substrate's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides coupled to respective particles) that are at different regions of an array may be differentiated from each other according to the locations of the regions in the array. An individual region of an array may include one or more molecules of a particular type. For example, a substrate region may include a single particle including amplicons of a target polynucleotide having a particular sequence. The regions of an array respectively may include different features than one another on the same substrate. Exemplary features include without limitation, wells in a substrate, particles (such as beads) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or hydrogel. Exemplary arrays in which separate substrates are located on a surface include, without limitation, those having particles (such as beads) in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A “partially” double stranded polynucleotide may have at least about 10%, at least about 25%, at least about 50%, at least about 60%, at least about 70%, at least about 80%, at least about 90% or at least about 95% of its nucleotides, but fewer than all of its nucleotides, hydrogen bonded to nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region that includes substantially only amplicons of a given polynucleotide may be referred to as “monoclonal,” while a substrate region that includes amplicons of polynucleotides having different sequences than one another may be referred to as “polyclonal.” A substrate region that includes a sufficient number of amplicons of a given polynucleotide to be used to sequence that polynucleotide maybe referred to as “functionally monoclonal.” Illustratively a substrate region in which about 60% or greater of the amplicons are of a given polynucleotide may be considered to be “functionally monoclonal.” Additionally, or alternatively, a substrate region from which about 60% or more of a signal is from amplicons of a given polynucleotide may be considered to be “functionally monoclonal.” A polyclonal region of a substrate may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide. The action by which a polynucleotide in solution becomes coupled to a substrate may be referred to as “capturing” the polynucleotide.

### Methods for Preparing Silica Particles

As provided herein, the present silica particles may be prepared using simple, cost-effective operations that may be used to control the particle size, as well as the functional groups that may be used to couple capture oligonucleotides to the particle for use in capturing and amplifying polynucleotides.

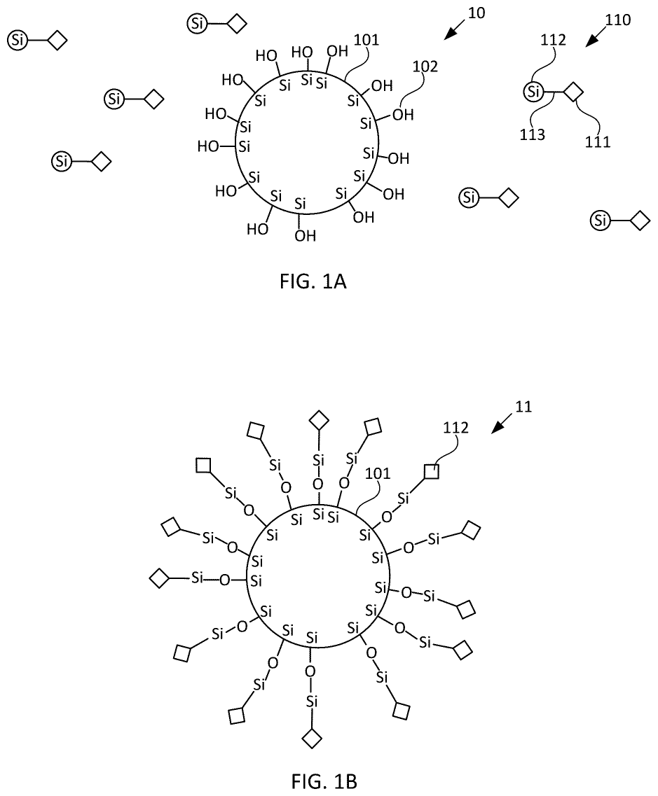

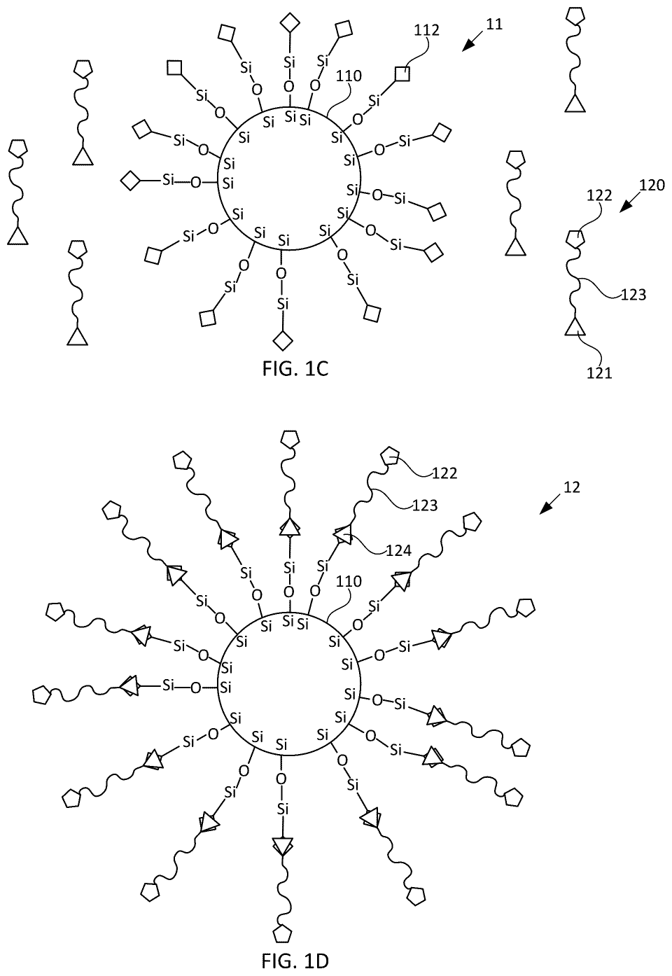

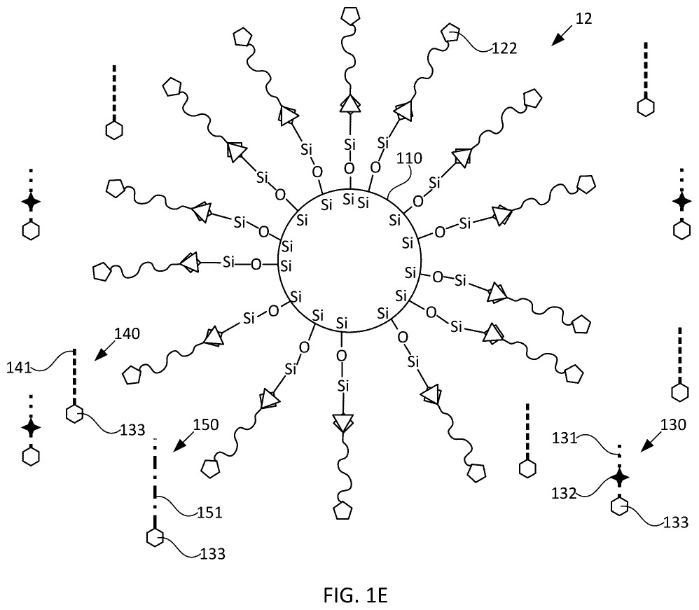

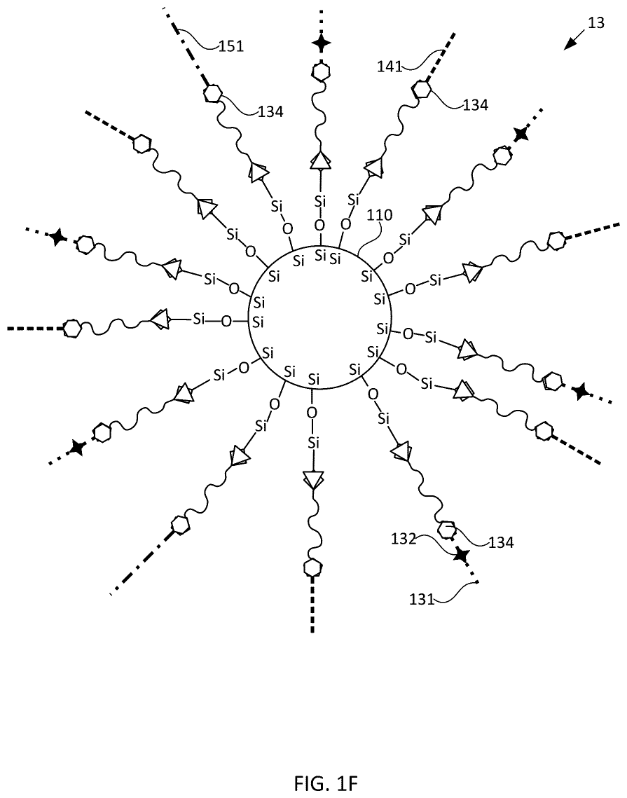

FIGS. 1A-IF schematically illustrate example structures and operations for preparing a silica particle coupled to a polymer, and optionally also to oligonucleotides. Referring first to FIG. 1A, the present silica particles may be prepared using a starting particle 10 that includes a core 101 the outer surface of which includes a plurality of silanol groups (—Si—OH) 102 as illustrated in FIG. 1A. In some examples, particle 10 may be prepared using the well-known Stöber process. For example, the reaction mix may include a silicate precursor, ammonium hydroxide, water, and an appropriate solvent. Nonlimiting examples of silicate precursors that may be used in the Stöber process include as silicic acid ([SiOₓ(OH)₄₋₂ₓ]ₙ), tetraethyl orthosilicate (TEOS), tetramethyl orthosilicate (TMOS), tetramethylsilane (TMS), dimethyldiethoxysilane (DMDES), polyethoxydisiloxane (PEDS), methyltriethoxysilane (MTES), ethyltriethoxysilane (ETES), tetrapropylorthosilicate (TPOS), (3-mercaptopropyl) trimethoxysilane (MPTMS), hexamethyldisiloxane (HMDSO), tetramethoxyvinylsilane (TMVS), sodium meta-silicate, tetrakis(2-hydroxyethyl) orthosilicate (THEOS) or any other silicon alkoxide precursor with the formula Si(OR)₄. Nonlimiting examples of solvents that may be used in the Stöber process include ethanol, methanol, propan-1-ol, isobutanol, butanol, tert-butyl alcohol, isopropanol alcohol (IPA), tert-amyl alcohol, benzyl alcohol, butanediol, tert-butyl alcohol, denatured alcohol, di(propylene glycol) methyl ether, diethylene glycol, ethylene glycol, 2-ethylhexanol, furfuryl alcohol, glycerol, 2-(2-methoxyethoxy) ethanol, methyl-butanol, 2-methyl-1-pentanol, neopentyl alcohol, 2-pentanol, 1,3-propanediol, propylene glycol, or propylene glycol methyl ether.

Within the mixture, the silicate precursor undergoes a series of hydrolysis and condensation reactions. For example, molecules of the silicate precursors may be hydrolyzed to form silanols (—Si—OH) that bond to other molecules of the silicate precursor to form a plurality of respective siloxane bridges (—Si—O—Si—). In a manner known in the art, the ammonium hydroxide provides an alkaline environment that directs the reactions towards the formation of substantially spherical silica particles such as illustrated in FIG. 1A; in the absence of such an alkaline environment, the silicate precursors may bond with one another to form a substantially linear polymer. Additionally, in a manner known in the art, the amount of water may be selected to adjust the size of the resulting particles 10. Optionally, particle 10 may have a diameter size ranging from about 10 nm to about 1 μm, e.g., about 50 nm to about 500 nm, e.g., about 100 nm to about 300 nm with low polydispersity index (PDI<1) (ISO 22,412:2017).

As illustrated in FIG. 1A, preparing the present silica particle may include contacting particle 10 with first molecules 110. Each of the first molecules 110 may include a silane 112 coupled to a first functional group 111. Silane 112 is illustrated in FIG. 1A as “Si” inside of a circle to represent that the Si may be bonded to one or more additional groups, such as one or more alkoxy groups, e.g., methoxy groups. Illustratively, silane 112 may include a trialkoxysilane group, such as a trimethoxysilane group. Functional group 111 may include any suitable group that may be used to couple a polymer to the silica particle in a manner such as will be described below. Silane 112 may be coupled directly to first functional group 111 via a covalent bond 113, or alternatively may be coupled to first functional group 111 via a linker such as will be described below. FIG. 1B illustrates particle 11 generated by reaction of particle 10 with a plurality of first molecules 110. Particle 11 includes first functional groups 112 coupled to core 101 via siloxane bridges (—Si—O—Si—). The process of forming these siloxane bridges, by bonding the Si of core 10 to the Si of silane 112, may be referred to as “silanization.” This silanization optionally may be carried out at any suitable time (e.g., immediately after) relative to the synthesis of the silica particles 10 without the need for any washing step, thus reducing the cost and complexity of preparing particles 11. For example, first molecules 110 may be added directly to the silica suspension resulting from formation of particle 10. The ammonium hydroxide remaining in the silica suspension may be used as a catalyst to facilitate the silanization. Particle 11 optionally may be solvent swapped into an aqueous solution, and any remaining ammonium hydroxide removed, e.g., by evaporation.

As illustrated in FIG. 1C, preparing the present silica particle may include contacting particle 11 with second molecules 120. Each of the second molecules 112 may include a polymer 123 coupled to a second functional group 121 and a third functional group 122. Although the second functional group 121 and third functional group 122 are illustrated for simplicity as being located at respective ends of polymer 123, it should be appreciated that these functional groups may have any suitable location relative to one another and relative to the polymer. In some examples, the polymer may include a polyacrylamide, such as PAZAM or SFA, that is synthesized in a manner as to include functional groups 121 and 122. Alternatively, the functional groups may be coupled to the polymer in a separate operation. In some examples, functional groups 121 and 122 may be of the same type as one another, while in other examples, functional groups 121 and 122 may be of different types than one another. Functional group 121 may be a partner pair to functional group 111. For example, functional group 111 may include a norbornene, a cyclooctyne, or a bicyclononyne, and functional group 121 may include an azide. For nonlimiting examples of pairs of functional groups which may be used to couple a silane to a polymer, see U.S. Pat. No. 10,975,210 to Berti et al., the entire contents of which are incorporated by reference herein. Functional group 122 may include the same type of group as functional group 121, or may include a different type of group. For nonlimiting examples of polymers including functional groups, see U.S. Pat. No. 10,975,210. FIG. 1D illustrates particle 12 generated by reaction of particle 11 with a plurality of second molecules 120. Particle 12 includes second functional groups 122 coupled to core 101 via polymer 123, via the reaction product 124 resulting from reaction of first functional group 111 and second functional group 121, and via the siloxane bridges formed in a preceding operation.

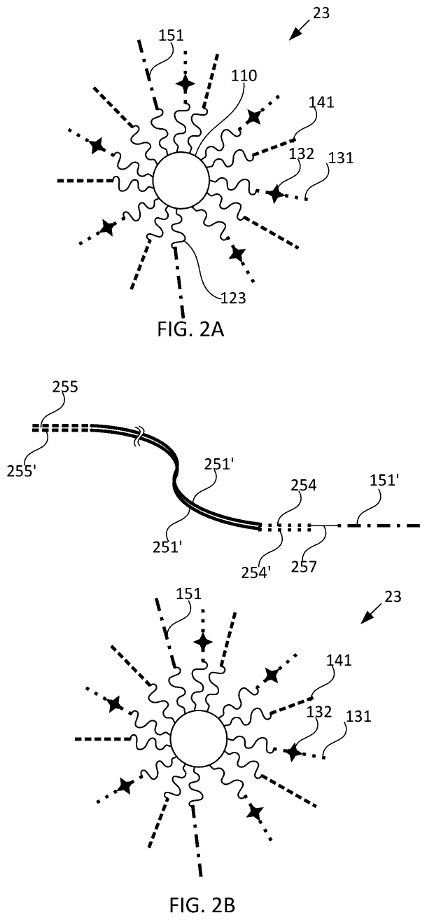

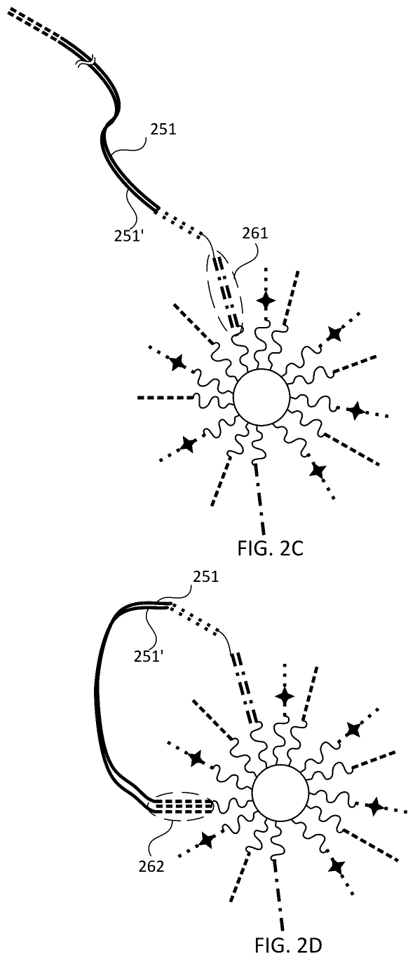

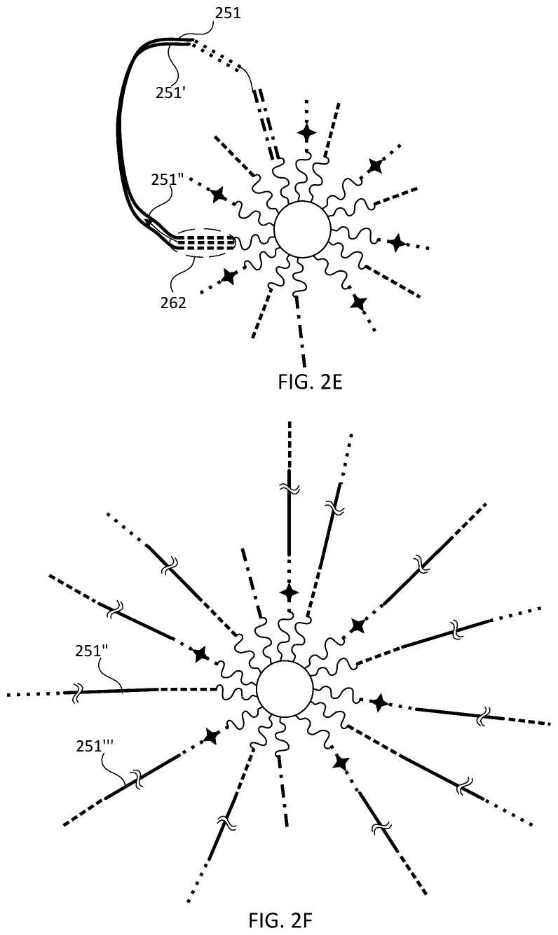

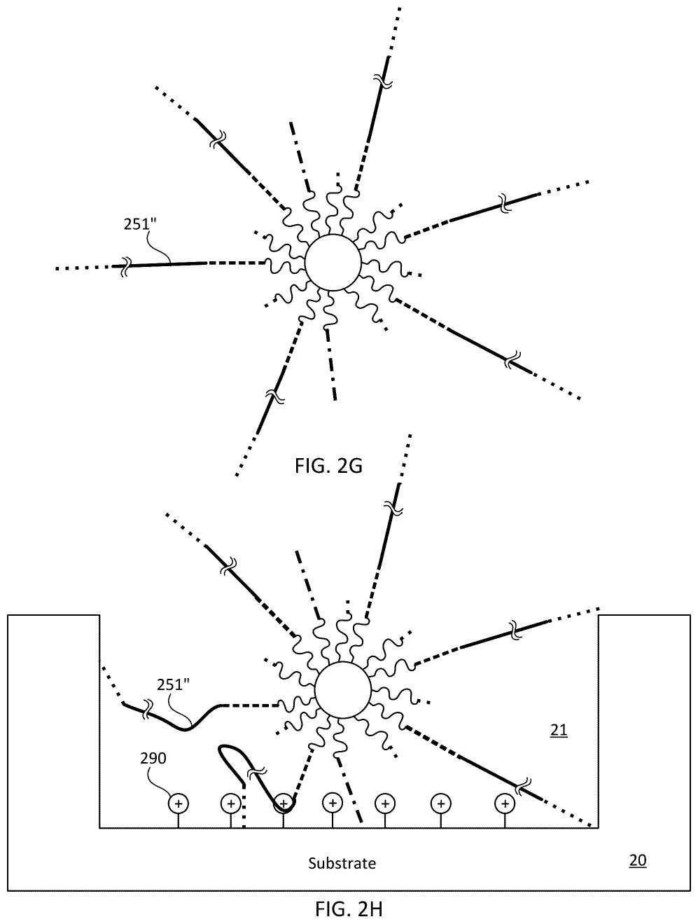

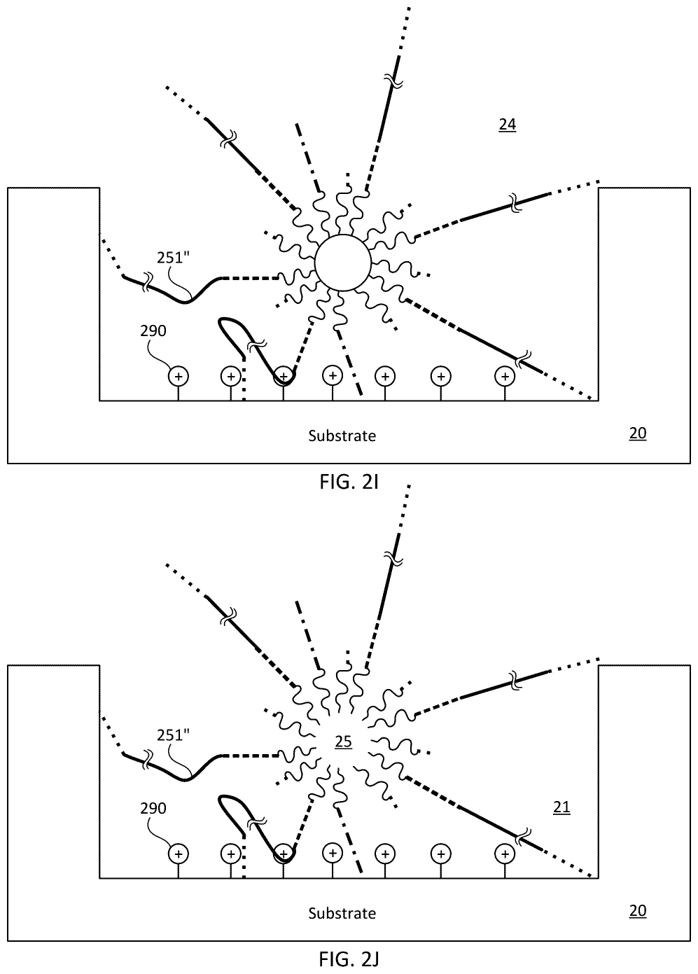

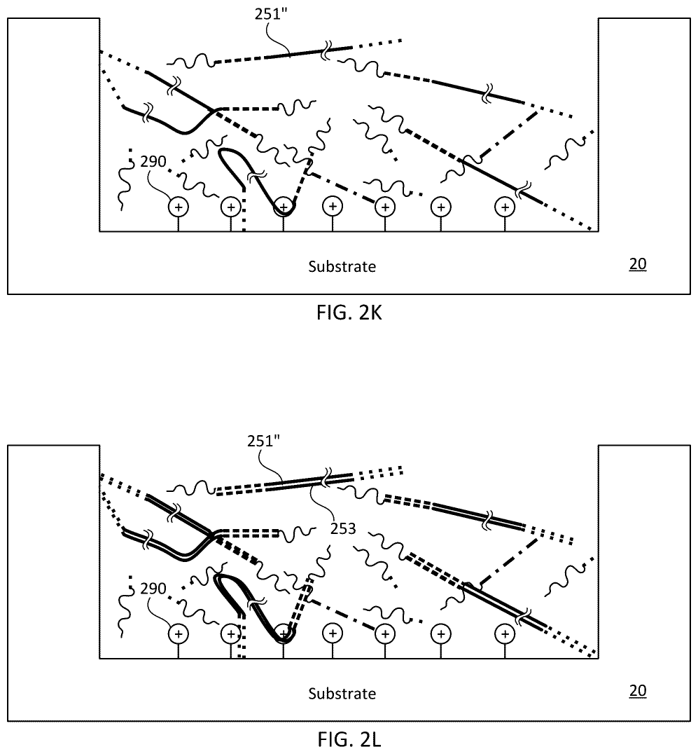

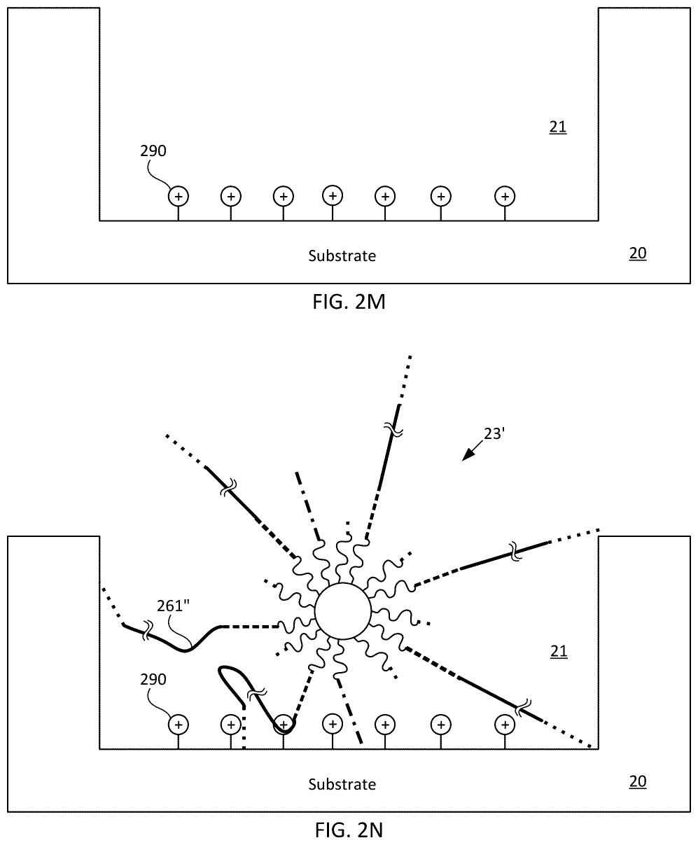

Optionally, oligonucleotides may be coupled to particle 12, e.g., so as to form a particle that optionally may be used in operations such as will be described with reference to FIGS. 2A-2N. For example, as illustrated in FIG. 1E, particle 12 may be contacted with third molecules each of which may include an oligonucleotide coupled to a fourth functional group 133. In the nonlimiting example illustrated in FIG. 1E, particle 12 is contacted with at least one of third molecules 130, 140, and/or 150, and optionally with a mixture of third molecules 130, 140, and/or 150. Molecules 130 may include a first oligonucleotide 131, which is coupled to fourth functional group 133 and optionally includes an excision moiety 132. Molecules 140 may include a second oligonucleotide 141 which is coupled to fourth functional group 133. Molecules 150 may include a third oligonucleotide 151 which is coupled to fourth functional group 133. The sequences of oligonucleotides 131, 141, and 151 may be orthogonal to one another. Illustratively, oligonucleotide 131 may be an amplification primer having the sequence P5 described further below, oligonucleotide 141 may be a different amplification primer having the sequence P7 described further below, and oligonucleotide 151 may be a capture primer having the sequence PX or PY described further below. FIG. 1F illustrates particle 13 generated by reaction of particle 12 with a plurality of third molecules, e.g., molecules 130, or molecules 140, or molecules 150, or a mixture of molecules 130 and 140, or a mixture of molecules 130 and 150, or a mixture of molecules 140 and 150, or a mixture of molecules 130, 140, and 150. As such, particle 13 includes oligonucleotides 131, 141, and/or 151 respectively coupled to core 101 via the reaction products 134 between third functional group 133 and second functional group 122, via polymer 123, via the reaction product 124 resulting from reaction of first functional group 111 and second functional group 121 in a preceding operation, and via the siloxane bridges formed in a preceding operation.

It will be appreciated that any suitable moieties may be used to couple elements to one another to form particle 13, e.g., to couple moiety 122 to moiety 133 to form reaction product 134. For example, such elements may be coupled to one another using covalent bonding selected from the group consisting of azide-alkyne bonding, transcyclooctene-tetrazine bonding, norbornene-tetrazine bonding, azide-cyclooctyne bonding, and azide-norbornene bonding.

A non-exclusive list of complementary binding partners that may be used to covalently couple moiety 122 to moiety 133 to form reaction product 134 is presented in Table 1. Note that moiety 121 and 122 can be of the same type as one another in certain examples, as explained above:

TABLE 1

| Bonding pair | Example moiety 122 | Example moiety 133 |
|---|---|---|
| azide-alkyne | azide, —N₃ | alkyne |
| | | 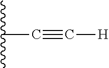 [SMILES: *C#C] |
| azide-cyclooctyne or azide- | azide, —N₃ | cyclooctyne, e.g. dibenzocyclooctyne |
| cyclononine | | (DBCO) |
| | | 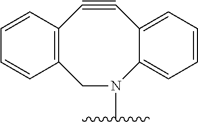 [SMILES: *N1Cc2ccccc2C#Cc2ccccc21] |
| | | or BCN (bicyclo[6.1.0]nonyne) |
| | | 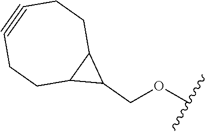 [SMILES: *OCC1C2CCC#CCCC21] |
| azide-norbornene | azine, —N₃ | norbornene |
| | | 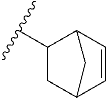 [SMILES: *C1CC2C=CC1C2] |
| transcyclooctene-tetrazine | tetrazine, e.g., benzyl- | transcyclooctene |
| | methyltetrazine | |
| | 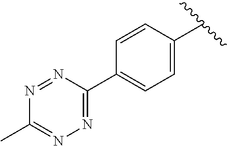 [SMILES: *c1ccc(-c2nnc(C)nn2)cc1] | 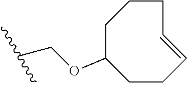 [SMILES: *COC1CC/C=C/CCC1] |
| norbornene-tetrazine | tetrazine, e.g., benzyl- | norbornene |
| | methyltetrazine | |
| | 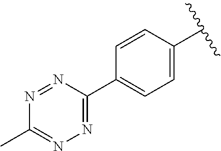 [SMILES: *c1ccc(-c2nnc(C)nn2)cc1] | 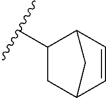 [SMILES: *C1CC2C=CC1C2] |

It is believed that particles 13 may be particularly well suited for use in capturing and amplifying polynucleotides, e.g., to generate substantially monoclonal clusters in a manner such as will be described below with reference to FIGS. 2A-2N. For example, the particles 13 may be used to generate the clusters in solution, which may facilitate the resulting clusters being substantially monoclonal. Additionally, it is believed that particles 13 may be particularly well suited for use in sequencing the amplified polynucleotides on a flowcell, in a manner that allows for the flowcell to be easily reused. For example, as will be described below with reference to FIGS. 2A-2N, the particles (with clusters coupled thereto) may be temporarily coupled to the flowcell during sequencing operations, and then may be easily removed after sequencing without damaging the flowcell, so that the flowcell may be reused. This may significantly reduce the cost of sequencing, as well as the amount of wasted material that otherwise may be generated during sequencing. Additionally, as will be described below with reference to FIGS. 2A-2N, the particle itself may be dissolved before or during the sequencing operations, such that the polymer and amplicons coupled thereto remain located within the flowcell during sequencing. Dissolving the particle may enhance the quality of the sequencing, for example by creating open space that facilitates contact between the sequencing reagents and the amplicons. After sequencing, the polymer and amplicons coupled thereto may be readily removed from the flowcell so that the flowcell may be reused.

### Methods for Capturing, Amplifying, and Sequencing Polynucleotides and Reusing Flowcells Using Particles

Some examples provided herein relate to using particles to capture a polynucleotide and then amplify that polynucleotide to generate a cluster which is at least functionally monoclonal, and in some examples is substantially monoclonal. The particle then may be disposed on a flowcell, the particle dissolved to leave the cluster on the flowcell, and the amplicons in the cluster then sequenced. The amplicons then may be removed from the flowcell, and the flowcell reused to sequence different amplicons which are coupled to a different particle.

For example, FIGS. 2A-2N schematically illustrate example structures and operations for capturing and amplifying a polynucleotide, and optionally also reusing a flowcell, using a particle that may be dissolved using suitable reagent(s), e.g., a silica particle. Referring first to FIG. 2A, particle 23 may include a plurality of each of first and second amplification primers 131, 141 which have orthogonal sequences to one another. In the nonlimiting example illustrated in FIG. 2A, particle 23 may include one or more seeding primers 151 which have an orthogonal sequence to that of amplification primers 131, 141 and which may be used to capture a polynucleotide having an adapter which is complementary to seeding primer 151 in a manner such as will be described with reference to FIGS. 2B-2C. In other examples, particle 23 may not include any seeding primers, and a sub-Poisson approach may be used to seed particles 23. More specifically, particles 23 may be contacted with a solution which contains a sufficiently dilute concentration of polynucleotides that each particle 23 is statistically likely to capture either one or zero polynucleotides from the solution. Particles 23 which capture one polynucleotide may then be used to amplify that polynucleotide to generate a cluster of amplicons, e.g., using bridge amplification, exclusion amplification (ExAmp), or other suitable amplification method, while a cluster of amplicons is not generated on particles which did not capture a polynucleotide. The particles with a cluster of amplicons have a different size, and a different charge, than particles without a cluster of amplicons. As such, the particles with the cluster of amplicons may be separated via enrichment based on size and/or charge from the particles without a cluster of amplicons.

Particle 23 may include a core 110 which may be dissolved using suitable reagent(s), e.g., a silica core, to which the seeding primer(s) 151 (if used) and amplification primers 131, 141 selectively may be coupled via polymer 123, e.g., using operations such as described with reference to FIGS. 1A-IF. In one nonlimiting example, capture primers 131 may include P5, capture primers 141 may include P7, and (if included) seeding primer(s) 151 may include PX or PY. In some examples, particle 23 may be configured substantially as described with reference to FIGS. 1A-1F, although it should be understood that particle 23 may be configured, and prepared, in a manner other than that described with reference to FIGS. 1A-1F.

At the particular time illustrated in FIG. 2A, particle 23 may be located within a fluid (fluid not specifically illustrated), such an aqueous buffer solution. In the nonlimiting example illustrated in FIG. 2B, particle 23 may be contacted, in the fluid, with double-stranded target polynucleotides, e.g., double-stranded polynucleotide fragments that are generated using commercially available fragmentation or tagmentation techniques, using DNA or RNA that it is desired to sequence. Although only one particle 23 is illustrated in FIG. 2B, it will be appreciated that the fluid may include thousands, or even millions, of particles that have substantially the same configuration as one another. Additionally, although only one double-stranded polynucleotide 251, 251′ is illustrated in FIG. 2B, it will be appreciated that the fluid may include thousands, or even millions, of polynucleotides which in some cases may have different lengths than one another. Each of the polynucleotides 251, 251′ may include first and second double-stranded adapters, e.g., first adapter 254, 254′ one strand of which is substantially complementary to amplification primer 131, e.g., is or includes cP5 (also referred to as P5′); and second adapter 255, 255′ one strand of which is substantially complementary to amplification primer 141, e.g., is or includes cP7 (also referred to as P7′). The double-stranded polynucleotides optionally may include an additional single-stranded seeding adapter which may be used to immobilize the polynucleotide to the particle during the seeding process. For example, as shown in FIG. 2B, polynucleotide 251, 251′ optionally may include single-stranded seeding adapter 151′ which is substantially complementary to seeding primer(s) 151, and which optionally is coupled to adapter 254 of polynucleotide 251 via linker 257. In some examples, optional seeding adapter 151′ may include cPX (also referred to as PX′) or cPY (also referred to as PY′). Note that because it is single-stranded, optional seeding adapter 151′ is available to hybridize to optional seeding primer 151 in a manner such as now will be described. Other methods for capture a polynucleotide using amplification primers 131, 141 (e.g., sub-Poisson methods) without the use of seeding primers and seeding adapters suitably may be used.

In nonlimiting examples including use of seeding primer 151 and seeding adapter 151′, optional linker 257 may be or include one or more of: a carbon-containing chain with a formula (CH₂)ₙ wherein “n” is from 1 to about 1500, for example less than about 1000, preferably less than 100, e.g. from 2-50, particularly 5-25; polyethylene glycol (PEG); or iSp9 (Spacer 9) which is a triethylene glycol spacer that can be incorporated at the 5′-end or 3′-end of an oligo or internally. Linkers formed primarily from chains of carbon atoms or from PEG may be modified so as to contain functional groups which interrupt the chains. Examples of such groups include ketones, esters, amines, amides, ethers, thioethers, sulfoxides, sulfones. Separately or in combination with the presence of such functional groups, groups such as alkene, alkyne, aromatic or heteroaromatic moieties, or cyclic aliphatic moieties (e.g., cyclohexyl) may be included. Cyclohexyl or phenyl rings may, for example, be connected to a PEG or (CH₂)ₙ chain through their 1- and 4-positions. Other linkers are envisaged which are based on nucleic acids or monosaccharide units (e.g., dextrose). It is also within the scope of this disclosure to utilise peptides as linkers. A variety of other linkers may be employed. The linker preferably is stable under conditions under which the polynucleotides are intended to be used subsequently, e.g., conditions used in DNA amplification. The linker should also be such that it is not by-passed by DNA polymerases, terminating DNA polymerization before copying optional adapter 151′ (if it is nucleotide based such as a PX′ sequence). This allows for the optional adapter 151′ to remain single-stranded and available for hybridization in a manner such as will now be described.

Referring now to the nonlimiting example shown in FIG. 2C, seeding adapter 151′ of double-stranded polynucleotide 251, 251′ may hybridize to seeding primer 151 to form duplex 261. Because adapters 254, 254′ and 255, 255′ are double-stranded, they are not available to hybridize to primers 131 and 141 respectively until after certain processing steps are performed, as will be explained below. Accordingly, substantially all seeding of the polynucleotide may be expected to be via hybridization between seeding adapter 151′ and seeding primer 151. After the initial hybridization described with reference to FIG. 2C, polynucleotide 251, 251′ may be amplified relatively quickly. More specifically, as illustrated in FIG. 2D, the double-stranded polynucleotide 251, 251′ may bend such that adapter 255, 255′ hybridizes with one of primers 141 to form duplex 262 using a process that may be referred to as “strand invasion” and may be promoted using a recombinase (not specifically illustrated in FIG. 2D). An amplified cluster then may be formed using polynucleotide 251, 251′. For example, FIG. 2E illustrates the composition of FIG. 2D during recombinase-promoted extension of the primer 141 to which double-stranded polynucleotide 251, 251′ hybridizes to form amplicon 251″ which is covalently coupled to the particle 23. Amplicon 251″ repeatedly may be further amplified using strand invasion. For example, it may be seen that the composition of FIG. 2F includes amplicon 251″ and a plurality of additional amplicons 251′″ of amplicon 251″ that are formed using a mixture of capture primers 131 and 141 for the amplification.

Amplification operations may be formed any suitable number of times so as to substantially fill the particle 23 with an at least functionally monoclonal cluster, and in some examples a substantially monoclonal cluster, e.g., with amplicons of target polynucleotide 251, 251′. For example, the amplicons coupled to the particle 23 may include at least about 60% amplicons of one selected target polynucleotide, or at least about 70% amplicons of one selected target polynucleotide, or at least about 80% amplicons of one selected target polynucleotide, or at least about 90% amplicons of one selected target polynucleotide, or at least about 95% amplicons of one selected target polynucleotide, or at least about 98% amplicons of one selected target polynucleotide, or at least about 99% amplicons of one selected target polynucleotide, or about 100% amplicons of one selected target polynucleotide. If amplification operations are repeated until the particle is substantially full, both adapters of the resulting amplicons may not necessarily be hybridized to corresponding capture primers or orthogonal capture primers, and as such the amplicons may extend linearly away from the particle 23 as illustrated in FIG. 2F. For further details regarding seeding and amplification operations using strand invasion and seeding adapters and seeding primers that are orthogonal to capture adapters and capture primers, see International Patent Application No. PCT/US2022/053002, filed on Dec. 15, 2022 and entitled “Orthogonal Hybridization,” the entire contents of which are incorporated by reference herein. Although FIGS. 2B-2G illustrate an example in which polynucleotide capture and amplification operations being performed in solution, it should be appreciated that in other examples, any suitable ones of such operations instead may be performed after disposing the particle on a substrate, e.g., within recess 21.

In some examples, certain capture primers and orthogonal capture primers may include non-nucleotide moieties. Such non-nucleotide moieties may include, but are not limited to, excision moieties via which a portion of the capture primers selectively may be removed. For example, capture primers 131 optionally may include excision moieties 132 and/or capture primers 141 optionally may include excision moieties (excision moieties of capture primers 141 not specifically shown in FIGS. 2A-2F or in FIGS. 1A-1F). The excision moieties 132 of capture primers 131 may be of the same type as, or a different type than, the excision moieties of capture primers 141. The excision moieties may be located at any suitable position along the length of any suitable primer(s) and may be, but need not necessarily be, the same type of excision moiety as one another. Following a desired number of amplification operations such as described with reference to FIGS. 2D-2F, portions of capture primers 131 may be removed by reacting a suitable enzyme or reagent with the respective excision moieties, and/or portions of capture primers 141 may be removed by reacting a suitable enzyme or reagent with the respective excision moieties. The enzyme or reagent used with the excision moieties of capture primers 131 may be the same as, or different than, the enzyme or reagent used with excision moieties of capture primers 141. Excision moieties 132 may be used to remove amplicons that are oriented in a selected direction, so that the remaining amplicons are oriented substantially in the same direction as one another in a manner such as illustrated in FIG. 2G.

In some examples, following the seeding and amplification operations, particle 23 with amplicons 251″ coupled thereto may be disposed within a flowcell and the amplicons sequenced, e.g., using sequencing-by-synthesis. Illustratively, as shown in FIG. 2H, a substrate 20 (e.g., a surface of a flowcell) may include a plurality of positively charged moieties 290. In the nonlimiting example illustrated in FIG. 2H, the substrate 20 includes a recess 21 in which the positively charged moieties 290 are disposed. Optionally, the positively charged moieties may be coupled to a hydrogel (not specifically illustrated) which is located on the substrate (e.g., within the recess 21). The amplicons 251″ may have a negative charge that is electrostatically attracted to the positively charged moieties 290. This electrostatic attraction may draw particle 23 and/or amplicons 251″ into contact with the substrate. Additionally, this electrostatic attraction and/or other force(s) may retain particle 23 and/or amplicons in contact with the substrate 20.

In some examples, after disposing particle 23 within the flowcell, the particle is dissolved, leaving the amplicons 251″ within the flowcell, and optionally also leaving polymer 123, to which the amplicons are coupled, within the flowcell. For example, as illustrated in FIG. 2I, a fluid 24 may be flowed into recess 21 that causes the core 110 of particle 23 to dissolve. Illustratively, fluid 24 may include any suitable reagent(s) to dissolve core 110, such as a basic solution, e.g., having a pH of about 12-13. The particle may be dissolved at any suitable time after disposing the particle within the flowcell. For example, the particle may be dissolved while using the flowcell to sequence the amplicons in a manner as will be described with reference to FIGS. 2K-2L; in such examples, fluid 24 may be a fluid that is used during one or more of such sequencing operations. Optionally, the particle may dissolve gradually during the sequencing operations. Alternatively, the particle may be dissolved before using the flowcell to sequence the amplicons. Any suitable combination of hardware and software components may be used to dissolve the particle. For example, the device in which the flowcell is located may include a reservoir storing a fluid comprising a reagent in a concentration suitable to substantially dissolve the particle, substantially without damaging the polymer and/or substantially without damaging the amplicons. The device further may include a fluidic pathway coupling the reservoir to the flowcell; and circuitry configured to cause the fluid to flow from the reservoir to the flowcell so as to contact, and substantially dissolve, the particle, leaving the polymer and/or amplicons within the flowcell.

In a manner such as illustrated in FIG. 2J, dissolving the core of particle 23 leaves additional empty space 25 within recess 21. Amplicons 251″ and polymer 123, which are no longer coupled to particle 23, may rearrange within recess 21, including within empty space 25. For example, as shown in FIG. 2K, amplicons 251″ and polymer 123, and other components which formerly were coupled to particle 23, may loosely occupy recess 21, leaving empty spaces into which fluid may flow. The electrostatic attraction which initially drew particle 23 into the recess 21, e.g., attraction between positively charged moieties 290 and negatively charged amplicons 251, may continue to retain the amplicons within the recess even in the absence of the particle itself. As such, amplicons 251″ and polymer 123 may remain within recess 21, even while fluids are flowed through recess 21 during sequencing operations.

The amplicons 251″ may be sequenced at any suitable time before or during dissolution of particle 23, e.g., using sequencing-by-synthesis operations that use reagents which may be flowed across the substrate 20. For example, sequencing-by-synthesis operations (not specifically illustrated) may generate, and determine the sequence of, amplicons 253 of amplicons 251″ as illustrated in FIG. 2L and thus may determine the sequence of amplicons 251″. After sequencing amplicons 251″, the polymer 123 and amplicons 251″, 253 may be removed. For example, in a manner similar to that described with reference to FIG. 2I, a fluid (not specifically illustrated) may be flowed into recess 21 that removes the amplicons 251″, 253, polymer 123, and any residual elements of particle 23 from recess 21. Illustratively, the fluid may include a basic solution, e.g., having a pH of about 13-14. Following removal of such components, the recess 21 may include positively charged moieties 290, and may be ready for reuse. For example, the recess may be used to sequence another particle 23′ coupled to another amplicon 261″ such as illustrated in FIG. 2N, in a manner such as described with reference to FIGS. 2H-2L. The process of removing particles and reusing the recess 21 (and the flowcell within the recess is disposed) may be repeated any suitable number of times.

From the foregoing disclosure, it will be appreciated that primers and adapters having any suitable sequences may be used. In one nonlimiting example, amplification primers 131 are P5 amplification primers, and amplification primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters 155 may be full-length complementary P7 adapters (cP7) having the sequence 5′-TCGTATGCCGTCTTCTGCTTG-3′ (SEQ ID NO: 4), and are commercially available from Illumina, Inc. Seeding primers 151 may have any suitable sequence which is orthogonal to the sequences of amplification primers 131, 141. In some examples, seeding primers 151 may be PX primers having the sequence AGGAGGAGGAGGAGGAGGAGGAGG (SEQ ID NO: 5). In other examples, seeding primers 151 may be PY primers having the sequence 5′-GAA GAA GAA GAA GAA GAA GAA GAA GAA GAA-3′ (SEQ ID NO: 6). In examples in which seeding primers 151 are PX primers, seeding adapters 151′ may be cPX (also referred to as PX′) adapters having the sequence CCTCCTCCTCCTCCTCCTCCTCCT (SEQ ID NO: 7). In examples in which seeding primers 151 are PY primers, seeding adapters 151′ may be cPY (also referred to as PY′) adapters having the sequence 5′-TTC TTC TTC TTC TTC TTC TTC TTC TTC TTC-3′ (SEQ ID NO: 8).

It will be appreciated that various examples herein may be used with operations consistent with “bridge amplification” or “surface-bound polymerase chain reaction” and/or with other amplification modalities. One such amplification modality is “exclusion amplification,” or ExAmp. Exclusion amplification methods may further facilitate the amplification of a single target polynucleotide per particle and the production of a substantially monoclonal population of amplicons on a particle. For example, the rate of amplification of the first captured target polynucleotide using the present particles may be more rapid relative to much slower rates of transport and capture of target polynucleotides using the present particles. As such, the first target polynucleotide captured by the particle may be amplified rapidly and fill the entire particle, thus further inhibiting the seeding and/or amplification of additional target polynucleotide(s) by the particle. Even if a second target polynucleotide is captured at the particle after the first polynucleotide, the relatively rapid amplification of the first polynucleotide may fill enough of the particle to result in a signal that is sufficiently strong to perform sequencing by synthesis (e.g., the second region may be at least functionally monoclonal). The use of exclusion amplification may also result in super-Poisson distributions of particles including monoclonal clusters; that is, the fraction of particles in a collection of particles that are functionally monoclonal may exceed the fraction predicted by the Poisson distribution. Increasing super-Poisson distributions of useful clusters is useful because more functionally monoclonal particles may result in higher quality signal, and thus improved SBS.

Another method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present particles and methods may be adapted for use with recombinase to facilitate the invasion of the present amplification primers and orthogonal amplification primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp). For still further examples of amplification methods that are compatible with the present particles, see International Patent Application No. PCT/US2022/053005 to Ma et al., filed Dec. 15, 2022 and entitled “Hybrid Clustering,” and International Patent Application No. PCT/EP2023/058307 to Ma et al., filed Mar. 30, 2023 and entitled “Paired-End Resynthesis Using Blocked P5 Primers,” the entire contents of each of which are incorporated by reference herein.

From the foregoing, it will be appreciated that the present particles may be used to capture and amplify polynucleotides in solution, during sample preparation, before disposing the particles onto the surface of a flowcell. This has the potential to increase the quality of sequencing data (e.g., via improved clonality) and speed (e.g., because amplicons may be generated outside of the sequencing instrument). Additionally, once captured on the flowcell, the clustered particles may reduce any non-specific binding interaction within hydrogel-coated wells underneath the particles. In addition, the clustering in suspension may reduce strand invasion and pad-hopping, improving sequencing data quality. Additionally, the present particles may be used to recycle the flowcell after sequencing. As will be clear from the working examples that follow, dissolving the core of a particle may be used to improve the quality of the sequencing data, and to increase the amount of fluorescence signal. For example, dissolving the particle core before or during sequencing may leave the amplified cluster non-covalently bonded to the flowcell (e.g., within a recess of the flowcell), and after sequencing the amplified cluster may be easily removed so that the flowcell may be reused. As such, the present particles may be used as disposable scaffolds for clustering and for transporting the cluster onto the flowcell prior to sequencing. It should be appreciated that the present particles equivalently may be used for amplification on-board the sequencing instrument.

In some examples, after the present particles have been used to transfer the cluster of amplicons into the flowcell (whether the amplicons are formed in solution or in the flowcell), their cores are no longer needed. Removing the particle cores is believed to be beneficial for sequencing, for example for any suitable combination of one or more of the following reasons:
More diffusible hydrogel: sequencing reagents may be expected to diffuse better into the polymer (e.g., polyacrylamide hydrogel), thanks to the reduced steric hindrance of the dissolving, or dissolved, core;
More efficient washes: any non-specific binding polynucleotides present on the surface of the core may be removed together with the core, increasing the signal-to-noise ratio;
Intensity signal may decrease more slowly during the sequencing run: decreases in signal intensity may be mitigated by the removal, and the consequent filling of empty space by the polymer (e.g., polyacrylamide hydrogel) to provide a more diffusible intensity;
Stronger intensity on Read 2: direct consequence of the two preceding points; and/or
Reusable flowcells: clusters of polynucleotides are non-covalently bonded to the flowcell, and may be easily removed and washed away when desired.

Accordingly, in some examples, the present particles may be used as a vector to transport the cluster (or the polymer bearing primers) within the flowcell and optionally into a nanowell within the flowcell. It is not strictly necessary that the cores of the particles remain after the clusters are captured within the wells. Indeed, removing the particle cores may facilitate cluster growth and/or SBS, for example by increasing the number of strands per cluster which are accessible to a polymerase. After dissolution of the core of the particles, the polymer/primers/clusters are not covalently bound to the wells. As now will be described in the Working Examples, the clusters nonetheless remain stable within the well (throughout long sequencing run), for example due to non-covalent polymer entanglement.

### WORKING EXAMPLES

The following examples are intended to be purely illustrative, and not limiting of the present claims.

### Example 1

Silica particles were prepared using the Stöber process. More specifically, two solutions were prepared:
1) Ethanol (99%) and TEOS; and
2) Ethanol (99%), deionized (DI) water, and ammonium hydroxide (25 wt %).

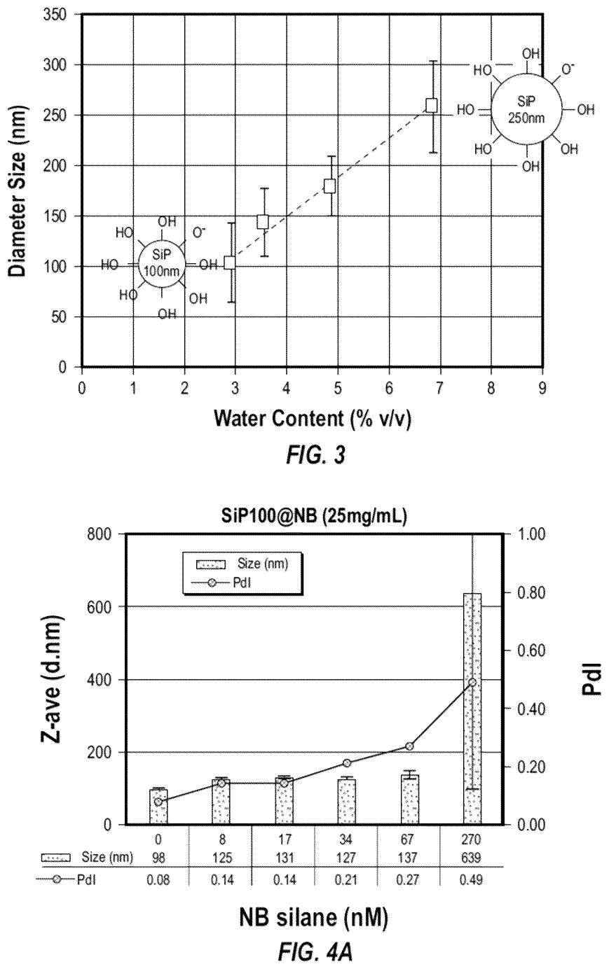

The TEOS was used as a precursor for the synthetic reaction described with reference to FIG. 1A. The two solutions were vigorously mixed together, the reaction was left for 24 hours at room temperature to form silica particles (SiPx). FIG. 3 illustrates a plot of the sizes, measured using dynamic light scattering (DLS), of example silica particles prepared according to this procedure with different percent water content in the mixture of the first and second solutions. More specifically, in FIG. 3, it may be seen that the diameter size of the silica particles generally increases as a function of water content. As shown in FIG. 3, silica particles having a diameter size of approximately 100 nm were prepared using a percent water content of about 3% (v/v); silica particles having a diameter size of approximately 140 nm were prepared using a percent water content of about 3.5% (v/v); silica particles having a diameter size of approximately 180 nm were prepared using a percent water content of about 4.8% (v/v); and silica particles having a diameter size of approximately 250 nm were prepared using a percent water content of about 6.8% (v/v). From this example, it may be understood that by appropriately selecting the percentage of water in the second solution, the average size of the silica particles made in a manner such as described with reference to FIG. 1A may be controlled. As such, it is expected that silica particles having a selected size in the range of about 10 nm to about 1 μm, e.g., about 50 nm to about 500 nm, or about 100 nm to about 300 nm, or about 100 nm to about 250 nm, may be readily prepared.

### Example 2

Silica particles having a diameter of approximately 100 nm were prepared according to Example 1. The surface of the silica particles was then silanized immediately after preparing the particles, without any washing step. More specifically, norbornene trimethoxysilane (NB-TMS), in varying concentrations, was added to 1 mL of the silica suspended in ethanol, in which the concentration of the particles in the suspension was 25 mg/mL. The ammonia within the suspension acted as a catalyst in the reaction scheme shown below:

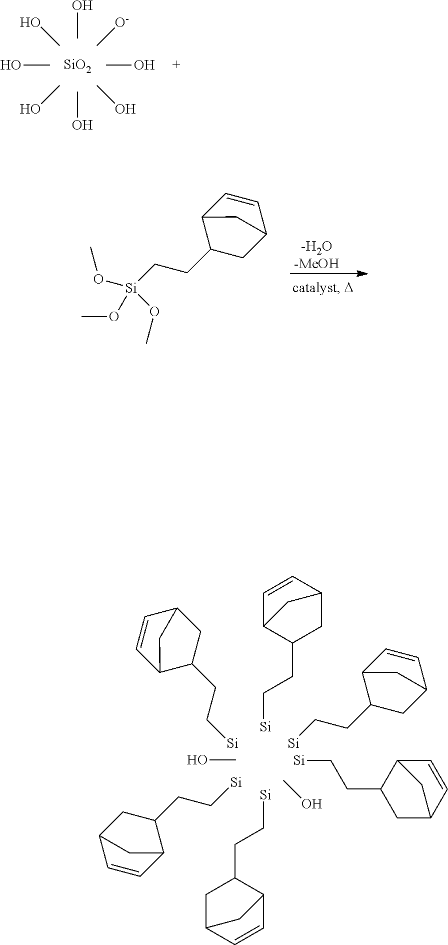

The mixture was sealed and incubated overnight (˜18 hours) at 60° C. A gentle mix of 350 rpm was used to homogenize the suspension. The silanized silica particles then were washed twice with DI water.

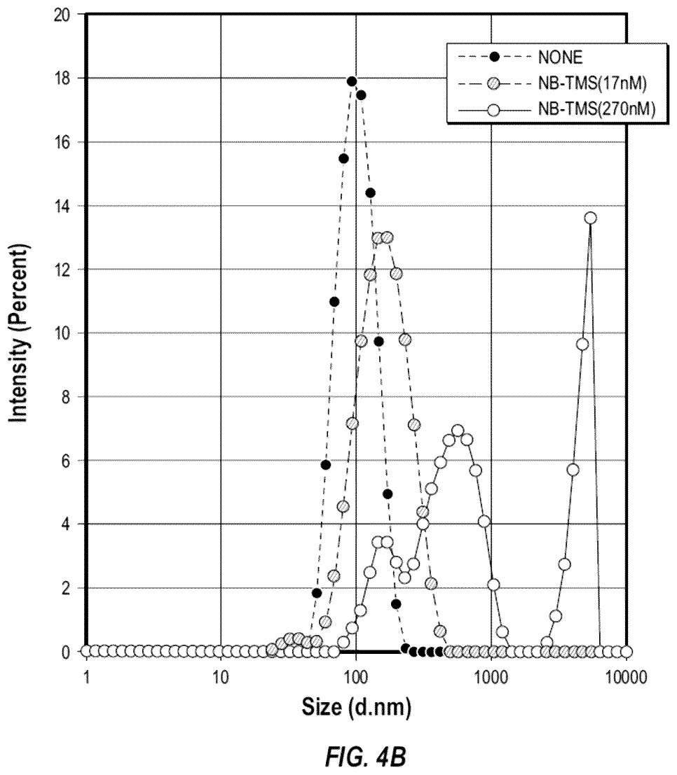

FIGS. 4A-4B illustrate plots of the measured size distributions of example silica particles prepared according to this example. More specifically, in FIG. 4A, bar graph plot 410 illustrates the average size of the particles after reaction with concentrations of NB-TMS ranging from 0 nM to 270 nM, and line plot 420 illustrates the polydispersity index (PdI) of these particles. From FIG. 4A, it may be understood that a concentration of 17 nM of NB-TMS was sufficient to substantially cover the surface of the silica particles. Higher concentrations of NB-TMS may be understood to have generated poorer quality samples (as reflected by a second peak in the dynamic light scattering (DLS, not shown) and/or higher polydispersity index), with presence of aggregates at the maximum amount of NB-TMS investigated, 270 nM (FIG. 4B). From this example, it may be understood that by appropriately selecting the concentration of silane molecules that are added to the particles in a manner such as described with reference to FIGS. 1A-1B, the coverage of the silane on the surface of the particle core may be controlled. As such, it is expected that silica particles which are silanized to include a coating of functionalized molecules may be readily prepared.

### Example 3

Silica particles having average diameters of about 120 nm or 200 nm coupled to norbornene groups were prepared according to Example 2, and then coupled to PAZAM hydrogel (respectively “SiP120” or “SiP200”) in a manner such as described with reference to FIGS. 1C-1D. More specifically, the hydrogel was provided at 3 wt % (viscous solution), diluted in DI water to 0.5 wt %, and then incorporated into the silanized silica suspension. The reaction was incubated for 1 hour at 60° C., and then washed with DI water. A mixture of alkyne-functionalized P5 and P7 primers (concentration 25 μM) was then grafted to the PAZAM hydrogel in a manner such as described with reference to FIGS. 1E-1F. More specifically, the PAZAM coated silica particles were incubated with an alkaline mixture of the P5 and P7 primers for 1-2 hours at 60° C.

Table 2 below shows DLS measurements carried out after the norbornene-coated silica particles were coated with PAZAM, and optionally also the mixture of P5 and P7 primers.

| | | NB- | | | | | Zeta | |
|---|---|---|---|---|---|---|---|---|
| | conc. | TMS | PAZAM | P5/P7 | Z-Ave_av | | Potential | |
| material | (mg/mL) | (uL) | (%) | incubation (h) | (d · nm) | Pdl_av | (mV) | stdev. |
| SiP120 | 50 | none | none | none | 104.0 | 0.14 | — | — |
| | 25 | 4 | none | none | 131.8 | 0.15 | −50.25 | 10.42 |
| | 5 | 4 | 0.5 | none | 227.1 | 0.09 | +1.19 | 3.70 |
| | 1 | 4 | 0.5 | 1.5 | 469.4 | 0.13 | −35.40 | 5.03 |
| SiP200 | 50 | none | none | none | 144.5 | 0.05 | — | — |
| | 25 | 4 | none | none | 167.4 | 0.05 | −65.12 | 19.43 |
| | 5 | 4 | 0.5 | none | 284.9 | 0.03 | −0.03 | 3.66 |
| | 1 | 4 | 0.5 | 1.5 | 575.9 | 0.02 | −34.20 | 4.35 |

From Table 2, it may be understood that the diameter size of the coated particles increased by a factor of approximately two when PAZAM was added, and even more significant was an increase in value of the Zeta potential from negative to zero (or slightly positive), as expected moving from a surface rich in negative charge (for norbornene silane) to a surface including azide groups (for PAZAM). From Table 2, it also may be understood that when the P5 and P7 primers were added, the diameter of the PAZAM coated particles further increased and the drop in Zeta potential was reestablished as a result of grafting the negatively charged primers, which increases the negative charge density at the surface of the particles. As such, from this example it may be understood that a polymer, such as a polyacrylate (e.g., PAZAM) successfully may be coupled to silica particles via functional groups that are coupled to the silica particles, in a manner such as described with reference to FIGS. 1C-1D. Additionally, from this example it may be understood that oligonucleotide primers optionally may be coupled to the polymer via functional groups that are coupled to the polymer, in a manner such as described with reference to FIGS. 1E-1F.

### Example 4

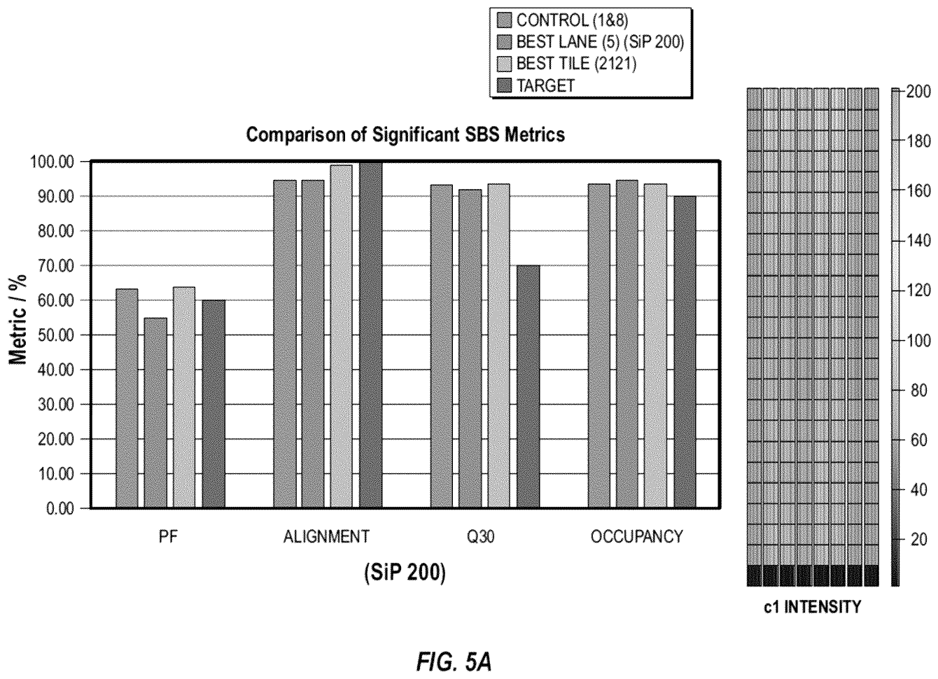

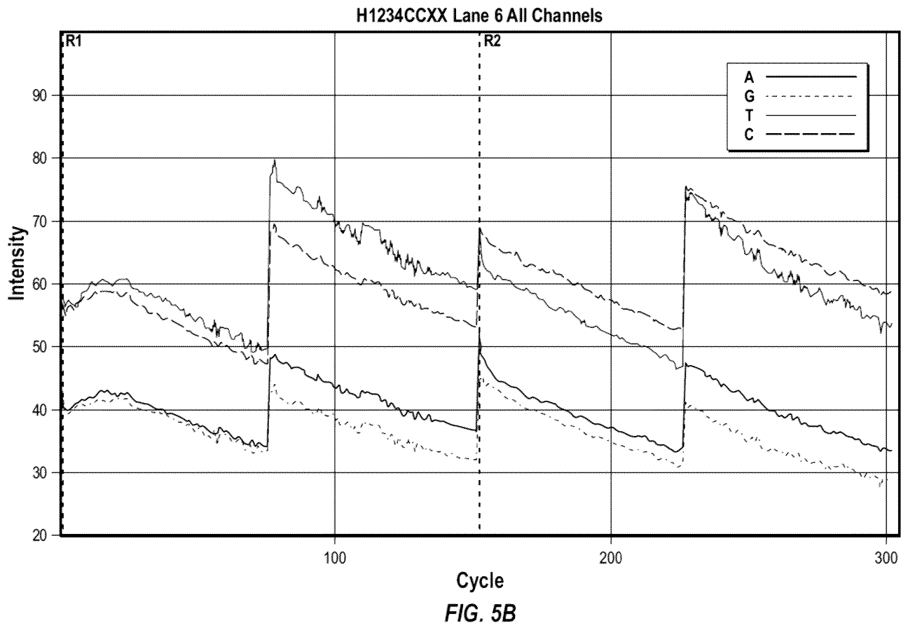

FIGS. 5A-5B illustrate fluorescence signals obtained from a flowcell during sequencing operations performed using example silica particles.

More specifically, silica particles having average diameters of about 100 nm or 200 nm coupled to PAZAM and grafted with P5 and P7 primers were prepared according to Example 3 (respectively “SiP120” or “SiP200”). The particles were disposed in lanes 2-7 of an eight-lane flowcell. Lanes 1 and 8 of the flowcell were used as positive control lanes (prepared with standard commercial surface chemistry including P5 and P7 primers grafted to PAZAM hydrogel pads on the flowcell surface). After the particles at different conditions were captured in the lanes of the flowcell, a cP5/cP7 CFR dye was flowed through the lanes of the flowcell. In detail, EtOH refers to the silanization step occurred in ethanol (water used if not specified). The x1 or x3 refers to the number of particles flushes used during the capturing step. The intensity of the fluorescent signal (“CFR Intensity”) was measured and is shown in FIG. 5A as well as Table 3 below. In Table 3, EtOH means that the silanization step has been carried out in ethanol (water otherwise), and x1 or x3 means that 1 or 3 flushes of silica particles have been used respectively. From FIG. 5A and Table 3, it may be understood that the density of clusters and the Cycle 1 intensity obtained using the present particles is comparable to the control. This demonstrates the usability of the present particles for SBS application with metrics equivalent to standard control.

TABLE 3

| | | CFR | Clusters | | Aligned | Intensity |
|---|---|---|---|---|---|---|
| Lane (#) | Sample ID | Intensity | PF (%) | % >= Q30 | (%) | Cycle 1 |
| 1 | control | 236 ± 20 | 61.8 ± 19.4 | 94.0 | 95.2 ± 20.0 | 435 ± 176 |
| 2 | SiP200_EtOH_x1 | 107 ± 19 | 54.4 ± 11.9 | 91.3 | 95.1 ± 19.9 | 90 ± 21 |
| 3 | SiP200_x1 | 85 ± 12 | 53.1 ± 11.7 | 90.7 | 95.1 ± 19.9 | 86 ± 21 |
| 4 | SiP200_EtOH_x3 | 103 ± 9 | 50.8 ± 11.2 | 88.9 | 94.9 ± 19.9 | 73 ± 19 |
| 5 | SiP200_x3 | 94 ± 4 | 55.3 ± 12.1 | 92.2 | 95.1 ± 19.9 | 98 ± 23 |
| 6 | SiP100_x3 | 116 ± 5 | 55.2 ± 12.1 | 92.4 | 95.2 ± 20.0 | 95 ± 22 |
| 7 | Commercial | 125 ± 6 | 42.2 ± 9.7 | 88.6 | 95.0 ± 19.9 | 71 ± 17 |
| | SiNPs_x3 | | | | | |
| 8 | control | 222 ± 17 | 65.6 ± 18.1 | 94.6 | 95.2 ± 20.0 | 401 ± 132 |

After dehybridization of the CFR dye, the flowcell was seeded with the PhiX library and clustered with a standard commercial clustering kit. The clusters then were sequenced on a HiSeq X instrument (Illumina, Inc.) using 1 read of 36 cycles. The primary metrics obtained are shown in Table 3, with the best tile reported in FIG. 5A. Despite lower fluorescence intensity compared to the control lanes 1 and 8, lanes 2-7 have good quality passing filter (“Clusters PF (%)”), Q30 data, and percent alignment after one cycle. Additionally, the signal intensity collected on another flowcell with similar workflow was stable throughout 300 read cycles (FIG. 5B). As such, it may be understood that the clusters remain suitably coupled to the substrate during the sequencing operations, although the silica cores may have been dissolving during these additional read cycles. Thus, this example demonstrates that silica particles prepared according to FIGS. 1A-1F suitably may be used for capturing, amplifying and sequencing polynucleotides, for example in a manner such as described with reference to FIGS. 2A-2L.

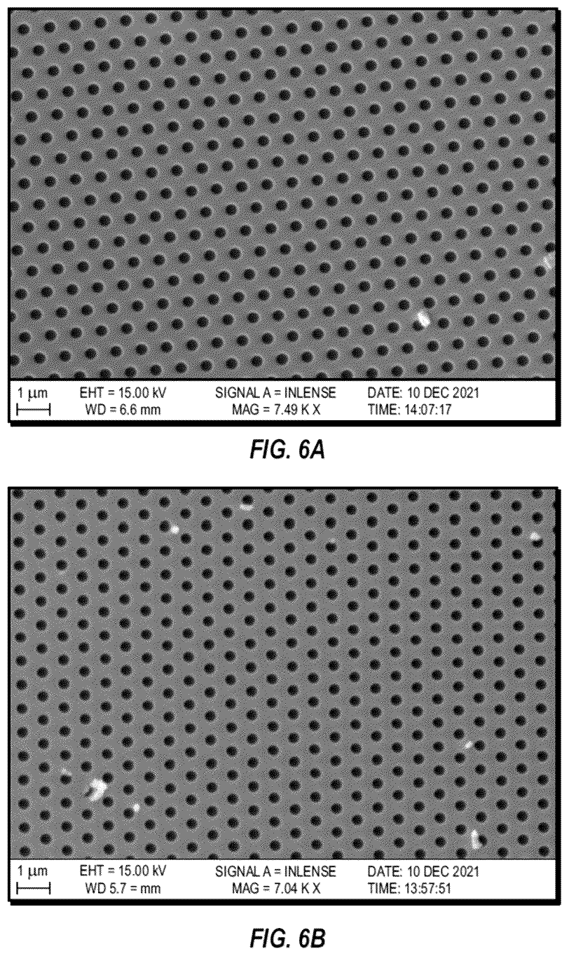

FIGS. 6A-6B respectively illustrate SEM images of the top and bottom of a flowcell after the above-described sequencing operations performed using the silica particles of the present example. No harsh chemicals were used to clean the surface and remove the beads. As may be seen in FIGS. 6A-6B, the recesses (nanowells) of the flowcell are empty of beads on both the top and bottom surfaces. This was observed across all lanes and conditions. The flowcell looks pristine on both surfaces. This indicates that the same flowcell may be reused to perform sequencing using additional particles, for example in a manner such as described with reference to FIGS. 2M-2N.

### Example 5

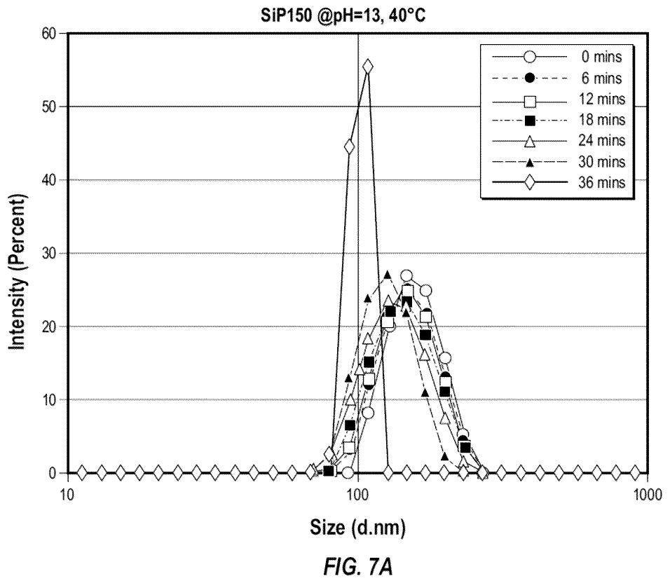

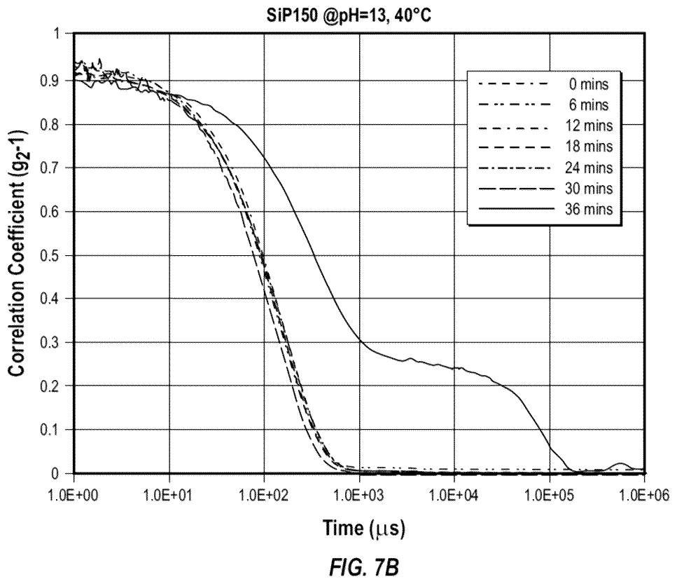

Bare silica particles prepared using Example 1, and having an average diameter of about 150 nm, were exposed to selected reagents that are used during sequencing operations such as described in Example 4. FIG. 7A illustrates a plot of the size distributions, measured using DLS, of example silica particles during exposure to a dissolving reagent, and FIG. 7B illustrates a correlogram of example silica particles during exposure to the dissolving reagent. More specifically, an initial concentration of 25 mg/mL of the silica particles was diluted in 0.1 M NaOH (pH=13) to obtain a final concentration of 1 mg/mL of the silica particles. After exposure to NaOH for 40 minutes at 40° C., no particles were detected. Indeed, the presence of a peak shown in FIG. 7A after 36 minutes can be associated to a spike as the estimated diameter size is of ˜700 nm rather ˜105 nm (mean of the “spike” peak). This assumption is corroborated by the correlogram shown in FIG. 7B. More specifically, considering that smaller particles move faster than bigger particles, and the exponential decay (autocorrelation function) is inversely proportional to the particles' motion, the decay of the particles exposed to high pH would be expected to occur in less time compared to the native particles. Instead, the decay of silica at high pH is delayed, confirming that the peak observed is a spike and should not be associated to particles of ˜105 nm in diameter size.

Thus, this example demonstrates that silica particles prepared according to FIGS. 1A-1F suitably may be dissolved in a manner such as described with reference to FIGS. 2I-2J.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of sequencing a polynucleotide, the method comprising:
hybridizing the polynucleotide to a first oligonucleotide coupled to a particle;
amplifying the hybridized polynucleotide using additional oligonucleotides coupled to the particle, to generate amplicons coupled to the particle;
after the amplifying, disposing the particle, having the amplicons coupled thereto, within a flowcell;
after disposing the particle within the flowcell, dissolving the particle, leaving the amplicons within the flowcell; and
using the flowcell to sequence the amplicons.

2. The method of claim 1, wherein disposing the particle, having the amplicons coupled thereto, within the flowcell comprises electrostatically attracting the particle or the amplicons to a region of the flowcell.

3. The method of claim 1, wherein the region of the flowcell comprises a recess.

4. The method of claim 3, wherein the recess comprises a hydrogel.

5. The method of claim 3, wherein the recess comprises a plurality of positively charged moieties.

6. The method of claim 1, wherein the particle is dissolved while using the flowcell to sequence the amplicons.

7. The method of claim 1, wherein the particle is dissolved before using the flowcell to sequence the amplicons.

8. The method of claim 1, wherein the oligonucleotides are coupled to a polymer that is coupled to the particle.

9. The method of claim 8, wherein dissolving the particle further leaves the polymer within the flowcell, the amplicons being coupled to the polymer left within the flowcell.

10. The method of claim 9, further comprising, after sequencing the amplicons, removing the polymer and the amplicons.

11. The method of claim 10, wherein the polymer and the amplicons are removed using a basic solution.

12. The method of claim 8, wherein the polymer is coupled to the particle via a siloxane (—Si—O—Si—) bond.

13. The method of claim 8, wherein the polymer comprises polyacrylamide.

14. The method of claim 8, wherein before the particle is dissolved, the polymer is covalently coupled to the particle.

15. The method of claim 8, wherein both before and after the particle is dissolved, the polymer is covalently coupled to the amplicons.

16. The method of claim 8, wherein both before and after the particle is dissolved, the polymer is non-covalently coupled to the flowcell.

17. The method of claim 1, wherein the particle comprises silica.

18. The method of claim 1, wherein the particle is dissolved using a basic solution.

19. (canceled)

20. A method of sequencing a polynucleotide using a particle having a plurality of oligonucleotides coupled thereto, the method comprising:
using the plurality of oligonucleotides to generate amplicons of the polynucleotide that are coupled to the particle;
disposing the particle, having the amplicons coupled thereto, within a flowcell;
after disposing the particle within the flowcell, dissolving the particle; and
using the flowcell to sequence the amplicons.

21. A method of using a flowcell, the method comprising:
disposing a first particle, having a first set of polynucleotide amplicons coupled thereto, within the flowcell;
dissolving the first particle, leaving the first set of polynucleotide amplicons within the flowcell;
using the flowcell to sequence the first set of polynucleotide amplicons;
after sequencing the first set of polynucleotide amplicons, removing the first set of polynucleotide amplicons from the flowcell; and
after removing the first set of polynucleotide amplicons from the flowcell, disposing a second particle, having a second set of polynucleotide amplicons coupled thereto, within the flowcell.

22-50. (canceled)


SEQUENCE LISTING (8 sequences)

SEQ ID NO: 1 - DNA, 20 bp, synthetic construct
  source 1..20: mol_type = other DNA; note = P5 amplification primer
aatgatacggcgaccaccga

SEQ ID NO: 2 - DNA, 21 bp, synthetic construct
  source 1..21: mol_type = other DNA; note = P7 amplification primer
caagcagaagacggcatacga

SEQ ID NO: 3 - DNA, 20 bp, synthetic construct
  source 1..20: mol_type = other DNA; note = complementary P5 adapter
tcggtggtcgccgtatcatt

SEQ ID NO: 4 - DNA, 21 bp, synthetic construct
  source 1..21: mol_type = other DNA; note = complementary P7 adapter
tcgtatgccgtcttctgcttg

SEQ ID NO: 5 - DNA, 24 bp, synthetic construct
  source 1..24: mol_type = other DNA; note = PX primer
aggaggaggaggaggaggaggagg

SEQ ID NO: 6 - DNA, 30 bp, synthetic construct
  source 1..30: mol_type = other DNA; note = PY primer
gaagaagaagaagaagaagaagaagaagaa

SEQ ID NO: 7 - DNA, 24 bp, synthetic construct
  source 1..24: mol_type = other DNA; note = cPX adapter
cctcctcctcctcctcctcctcct

SEQ ID NO: 8 - DNA, 30 bp, synthetic construct
  source 1..30: mol_type = other DNA; note = cPY adapter
ttcttcttcttcttcttcttcttcttcttc